(19) United States
(12) Patent Application Publication
(10) Pub. No.: US 2009/0191194 A1
(43) Pub. Date: Jul. 30, 2009

(54) INHIBITION OF PROTEIN KINASE C ALPHA FOR THE TREATMENT OF CARDIOVASCULAR DISEASES

(75) Inventors: Jan MENNE, Hannover (DE); Hermann Haller, Hannover (DE)

Correspondence Address: VENABLE LLP, P.O. BOX 34385, WASHINGTON, DC, 20043-9998, US

(73) Assignee: Phenos GmbH, Hannover (DE)

(21) Appl. No.: 12/325,930

(22) Filed: Dec. 1, 2008

Related U.S. Application Data
(63) Continuation of application No. 10/528,806, filed on Jun. 14, 2005, now abandoned.

(30) Foreign Application Priority Data
Sep. 24, 2002 (DE) 102 44 453.6

(51) Int. Cl. A61K 31/395; A61K 31/711; A61K 31/7105; A61K 35/12; A61K 31/355; A61K 38/13

(52) U.S. Cl. 424/133.1; 514/44; 424/93.21; 424/158.1; 424/146.1; 514/458; 514/11

(57) ABSTRACT

The invention relates to the use of agents impeding the expression and/or activity of protein kinase C-alpha (PKC-α), especially for treatment of patients with diabetes and complications such as diabetic nephropathy, retinopathy or neuropathy.

The present invention relates to the use of agents which reduce or inhibit the expression and/or activity of protein kinase C-α (PKC-α) for the treatment and/or prevention of coronary heart disease, myocardial infarction, peripheral occlusive disease, stroke, renal diseases involving proteinuria, diabetic late effects and/or cardiovascular complications in patients with diabetes mellitus, cardiovascular complications in patients with hypertension, and cardiovascular complications in patients with hypercholesterolemia.

Diabetes mellitus is one of the most frequent diseases in the Western world and afflicts about 5% of the population. Diabetes mellitus is subdivided into diabetes type I, which usually occurs already in the youth, and diabetes type II, which is also referred to as adult-onset or maturity-onset diabetes. Due to a disorder in the glucose metabolism, permanently increased blood glucose levels occur in both diabetes forms, which results in different complications in the afflicted patients after several years. The most frequent and at the same time most feared complications are diabetic retinopathy, which results in blindness, diabetic neuropathy, which may lead to foot or leg amputations, and diabetic nephropathy.

Diabetic nephropathy will develop in about 400% of all diabetes patients and is the most frequent cause of chronic renal failure and dialysis treatment worldwide. About 30 to 40% of all new dialysis patients exhibit diabetic nephropathy. Since diabetic renal damage develops slowly, the early identification of patients who have an increased risk of developing renal insufficiency is of great clinical importance for initiating suitable therapeutic steps. One of the first clinical signs of beginning renal damage is the occurrence of a so-called microalbuminuria. This involves the excretion of 30-300 mg of albumin in 24 hours collected urine. Normally, less than 30 mg of albumin is excreted per day. Under the current therapeutic conditions, microalbuminuria will occur in about 25% of diabetics with diabetes type I or type II (Alzaid, Diabetes Care, 19: (1996), 79-89; Klein et al., Diabetes Care, 22 (1999), 743-751; Valjnadrid et al., Arch. Intern. Med., 160 (2000), 1093-1100). The risk that renal insufficiency develops is about 10 times higher in patients with microalbuminuria as compared to patients with normal albumin excretion. Diabetic nephropathy, which is characterized by a proteinuria of more than 300 mg/day and/or restricted renal function, will develop in about 5 to 10% of all patients with diabetes and microalbuminuria per year. The risk that diabetic retinopathy will develop is also significantly increased in diabetics with microalbuminuria as compared to diabetics without microalbuminuria (Vigstrup and Mogensen, Acta Opthalmol. (Copenh), 63 (1985), 530-534).

As shown in long-term studies with more than ten years of follow-up, cardiovascular mortality is increased about twice in type II and type I diabetics already in the stage of microalbuminuria as compared to diabetics without microalbuminuria, also after correction for conventional risk factors, such as cholesterol and hypertension (Rossing et al., Bmj, 313 (1996), 779-784; Gerstein et al., Diabetes Care, 23 (2000), Suppl. 2: B35-39; Valmadrid et al., 2000). An increased cardiovascular mortality can also be detected in patients with microalbuminuria without diabetes mellitus (Gerstein et al., Jama, 286 (2001), 421-426).

There are various hypotheses of why microalbuminuria is an extremely important marker for the development of complications in patients with diabetes. According to the so-called “steno hypothesis” (Deckert, Feldt-Rasmussen et al., Diabetologia, 32 (1989), 219-226), the loss of negatively charged, i.e., anionic, molecules in the extracellular matrix is responsible for the formation of albuminuria, diabetic retinopathy and cardiovascular complications, for example, coronary heart disease. This hypothesis is supported by data acquired in both humans and animal model systems, and has been confirmed in recent years by results obtained by other working groups.

In the kidney, the urine is secreted in the renal corpuscles, the so-called glomeruli. To prevent the passage of proteins, for example, albumin, the blood side is separated from the urine side by a membrane referred to as the basal membrane. The basal membrane has small pores which allow smaller molecules to pass through the basal membrane while protein molecules cannot pass the membrane due to their size. In patients with microalbuminuria, the passage of small proteins, such as albumin, nevertheless occurs although the pore size is not increased at first. To account for this phenomenon, it could be shown that molecules with negative charge which repel the also negatively charged proteins are present in the pores or at the edge of the pores (Kverneland, Feldt-Rasmussen et al., Diabetologia, 29 (1986), 634-639; Deckert, Feldt-Rasmussen et al., Kidney Int., 33 (1988), 100-106; Kverneland, Welinder et al., Diabetologia, 31 (1988), 708-710). These molecules with negative charges are proteoglycans. Proteoglycans are complex macromolecules which consist of proteins to which polysaccharide chains are covalently associated. The polysaccharide chains predominantly consist of heparan sulfate and have a high negative charge. The proteoglycan which occurs most frequently in the body is perlecan. Perlecan is a protein of 460 kD and has several polysaccharide side chains (Murdoch and Iozzo, Virchows Arch. A. Pathol. Anat. Histopathol., 423 (1993), 237-242; Iozzo, Cohen et al., Biochem. J., 302 (1994), 625-639; Murdoch, Liu et al., J. Histochem. Cytochem., 42 (1994), 239-249). In patients with diabetes and microalbuminuria, heparan sulfate is hardly present in the glomerular basal membrane. Also in patients with advanced diabetic nephropathy, heparan sulfate can no longer be detected in the basal membrane, even though the protein chains are still present. This effect is accounted for by the fact that heparan sulfate synthesis is reduced under hyperglycemic conditions, as occur in diabetics (Parthasarathy and Spiro, Diabetes, 31 (1982), 738-741; Deckert, Feldt-Rasmussen et al., 1988; Nakamura and Myers, Diabetes, 37 (1988), 1202-1211; Nerlich and Schleicher, Am. J. Pathol., 139 (1991), 889-899; Makino, Ikeda et al., Nephron, 61 (1992), 415-421; Scandling and Myers, Kidney Int., 41 (1992), 840-846; Vernier, Steffes et al., Kidney Int., 41 (1992), 1070-1080; Tamsma, van den Born et al., Diabetologia, 37 (1994), 313-320; Iozzo and San Antoniom, J. Clin. Invest., 108 (2001), 349-355). Further, It could be shown that heparan sulfate proteoglycans not only prevent the glomerular filtration of albumin by their negative charge, but are probably also responsible for the integrity of the pore size within the basal membrane (Deckert, Kofoed-Enevoldsen et al., Diabetologia, 36 (1993), 244-251). Thus, as the renal insufficiency proceeds, the loss of heparan sulfate proteoglycans results in a destruction of the microstructure of the basal membrane. These changes could explain why a great non-selective proteinuria with loss of larger proteins, such as immunoglobulin, occurs in the course of diabetic nephropathy. Heparan sulfate proteoglycans are also strong inhibitors of mesangial expansion in the renal corpuscle. This is of great interest since an expansion of the mesangial connective tissue classically occurs in diabetes patients. Therefore, it is not surprising that the loss of heparan sulfate proteoglycan in diabetes patients is accused as an important cause of mesanglal expansion.

However, the loss of heparan sulfate in diabetics occurs not only in the kidney, but in almost all other organs. Thus, there is a clear reduction of heparan sulfate in the connective tissue of the retina, the skeletal muscle, the arterial walls and the skin as well as on red blood cells. Endothelial cells also exhibit a reduced synthesis of heparan sulfate (Yokoyama, Hoyer, et al., Diabetes, 46 (1997), 1875-1880; van der Pijl, Daha et al., Diabetologia, 41 (1998), 791-798). Since heparan sulfate proteoglycans have important antithrombotic properties, the loss of heparan sulfate proteoglycans can contribute to the formation of microthrombi, for example, in the retinal vessels, and thus promote the formation of diabetic retinopathy (Marcum, Fritze et al., Am. J. Physiol., 245 (1983), H275-33; Marcum, McKenney et al., J. Clin. Invest., 74 (1984), 341-350; Marcum and Rosenberg, Biochemistry, 23 (1984), 1730-1737; Marcum, Atha et al., J. Biol. Chem., 261 (1986), 7507-7517). Further important anti-atherosclerotic functions of heparan sulfate proteoglycans (HSPG) include the inhibition by HSPG of the proliferation of vascular smooth muscle cells, which results in the formation of arterial vascular lesions. HSPGs further inhibit the binding of monocytes (inflammatory cells) to the subendothelial connective tissue. HSPGs also inhibit the subendothelial binding and deposition of lipoprotein a and oxidize LDLs, which play a critical role in the formation of atherosclerosis. HSPGs are also important regulators in angiogenesis, i.e., in the formation of new vessels in damaged body regions (Rosenberg, Shworak et al., J. Clin. Invest., 100 (1997), p. 67-75; Pillarisetti, Trens Cardiovasc. Med., 10 (2000), 60-65; Iozzo and San Antonio, 2001). Therefore, the loss of heparan sulfate proteoglycan is important not only to the development of diabetic nephropathy and diabetic retinopathy, but also in the development of cardiovascular complications.

A further aspect is the fact that microalbuminuria will occur in patients with hypertension. To date, this phenomenon has been explained by an increased pressure in the renal corpuscles, assuming that albumin is increasedly secreted. However, if this is the case, it must be considered that patients with a constantly high arterial blood pressure also have a high cardiovascular risk, irrespective of whether they exhibit microalbuminuria. However, this is not the case, as could be shown in several prospective studies. Hypertensive patients with microalbuminuria show a cardiovascular morbidity and mortality which is about twice as high as that of similarly hypertensive patients with otherwise comparable risk profile, for example, hypercholesterolemia, smoking history and diabetes (Sleight, J. Renin Angiotensin Aldosterone Syst., 1 (2000), 18-20; Crippa, J. Hum. Hypertens., 16 (Suppl. 1) (2002), p. 74-7; Diercks, van Boven et al., Can. J. Cardiol., 18 (2002), 525-535). Accordingly, microalbuminuria is an independent risk parameter of the development and prognosis of cardiovascular diseases. This can be explained only by the fact that a change in the whole vascular system occurs in patients with microalbuminuria. However, to date, it has been unclear which disorder in patients with hypertension is the basis of microalbuminuria.

The object of the invention is to provide agents which can be employed for the therapy of microalbuminuria, especially in patients with diabetes mellitus and patients with hypertension in order to treat and/or prevent the late effects associated with diabetes, especially diabetic retinopathy, diabetic nephropathy and diabetic neuropathy, and cardiovascular complications as well as the cardiovascular complications associated with hypertension.

The present invention achieves this object by using agents which reduce or inhibit the expression and/or activity of protein kinase C-α (PKC-α) for the treatment and/or prevention of vascular diseases, cardiovascular diseases, renal diseases involving proteinuria, diabetic late effects and/or cardiovascular complications in patients with diabetes mellitus, cardiovascular complications in patients with hypertension, and/or cardiovascular complications in patients with hypercholesterolemia.

In the prior art, it has been supposed to date that the β2 isoform of protein kinase C is responsible for the development of the diabetic complications. On the one hand, the β2 isoform is produced at an increased level in the tissue of diabetic animals (Inoguchi et al., Proc. Natl. Acad. Sci. USA, 89 (1992), 11059-11063), and on the other hand, the protein kinase C-p specific inhibitor LY333531 results in a reduced proteinuria as a sign of reduced renal damage in rodents with type I and type II diabetes (Ishii et al., J. Mol. Med., 76 (1998), 21-31; Koya et al., Faseb J., 14 (2000), 439-447).

Protein kinase C-α “knock out” mice produced according to the invention which are not able to form protein kinase C-α surprisingly did not develop albuminuria after the induction of diabetes by means of streptozotozin. In contrast, control animals which were genetically substantially identical except for the change of protein kinase C-α expression developed a clear albuminuria. According to the invention, the further examination of the “knock out” animals showed completely surprisingly that the animals were able to form heparan sulfate at a normal level under diabetic conditions. In contrast, the control animals were hardly able to form heparan sulfate any longer under diabetic conditions.

Histological examinations performed according to the invention resulted in further significant changes in the protein kinase C-α “knock out” mice. According to the invention, using immunohistochemical methods, it could be shown that the lack of protein kinase C-α entrains further significant differences in the expression of VEGF (vascular endothelial growth factor) and the related receptor (VEGF-R II). While a significant increase of the expressed amounts of VEGF and VEGF-R II receptor could be detected in diabetic control animals, a significantly lower increase of the expressed amounts of VEGF and VEGF-R II receptor was established in the protein kinase C-α “knock out” animals. This result is of immense importance because increased VEGF expression is considered one of the most important mediators for the development of diabetic retinopathy (Aiello and Wong, Kidney Int. Suppl., 77 (2000), p 113-9; Benjamin, Am. J. Pathol., 158 (2001), 1181-1184).

From the results according to the invention, it can be seen that protein kinase C-α plays a key role in the regulation of the heparan sulfate proteoglycan formation and in the manifestation of proteinuria. The results according to the invention also show that protein kinase C-α plays a significantly more important role in the manifestation of proteinuria as compared to protein kinase C-β, wherein protein kinase C-β is evidently capable of taking over at least part of the functions of protein kinase C-α, however. The results according to the Invention further show that an inhibition of the protein kinase C-α Isoform selectively offers protection from both the development of diabetic late effects, such as diabetic nephropathy, diabetic retinopathy and/or cardiovascular complications, and the development of diseases which are accompanied by proteinuria.

Thus, according to the invention, there is provided the use of agents which reduce or inhibit the expression and/or activity of protein kinase C-α (PKC-α) for the treatment and/or prevention of vascular diseases, cardiovascular diseases, renal diseases involving proteinuria, diabetic late effects and/or cardiovascular complications in patients with diabetes mellitus, cardiovascular complications in patients with hypertension and/or cardiovascular complications in patients with hypercholesterolemia.

In the context of the present invention, “diseases” refers to disorders of the vital processes in organs or in the whole organism which result in subjectively felt or objectively detectable physical, psychic or mental changes. “Complications” or “late effects” means consequential diseases or secondary diseases, i.e., a second disease which occurs in addition to a primary clinical picture.

According to the invention, the diseases to be treated are, in particular, vascular diseases, cardiovascular diseases, renal diseases involving proteinuria, diabetes mellitus with and without associated late effects and/or cardiovascular complications, hypertension with and without associated cardiovascular complications and/or hypercholesterolemia with and without associated cardiovascular complications.

In the context of the present invention, “vascular diseases” means, in particular, diseases of the arteries which may lead to functional or organic circulatory disturbance. In a preferred embodiment of the invention, the vascular disease is a peripheral arterial occlusive disease. “Arterial occlusive disease” means a disease which is caused by stenosing or obliterating changes in the arteries and results in circulatory disturbance with ischemia in tissues or organs which depend on supply. Diabetes mellitus, in particular, results in chronic occlusive diseases which are caused, inter alia, by obliterating atherosclerosis and also angiopathies and angioneuropathies.

“Cardiovascular diseases” means diseases and disorders which affect the function of the heart and circulation, for example, the filling state and tonus of the circulatory system, the output performance of the heart, the neural and humoral coupling mechanisms between the heart and circulation etc. In a preferred embodiment of the invention, the cardiovascular diseases are coronary heart disease, myocardial infarction and stroke.

“Coronary heart disease” means the clinical manifestation of a primary coronary insufficiency in which the constriction or occlusion of coronary vessels results in a reduction of circulation and thus the supply of energy-delivering substrates and oxygen to the cardiac muscle.

“Myocardial infarction” means the necrosis of a localized region of the cardiac muscle which mostly occurs acutely as a complication in chronic coronary heart disease. The cause of myocardial infarction is a continuing critical circulation deficiency in coronary insufficiency and extended coronary spasms, especially in the region of a pre-existing eccentric coronary stenosis. A myocardial infarction is often manifested upon physical or psychic stress as a consequence of an increased oxygen demand of the cardiac muscle or upon an acute interruption of the blood supply.

“Stroke” or “apoplexy” means an ischemic cerebral infarction as a consequence of arterial circulation disorders of the brain. Stroke is caused by embolisms derived from atherosclerotic changes in extracranial vessels or from the heart, less frequently as a consequence of stenosis or cerebral microangiopathies.

In the context of the present invention, “renal diseases involving proteinuria” means, in particular, parenchymal kidney diseases which are characterized by the presence of proteins in the urine. The proteinuria may be glomerular proteinuria, tubular proteinuria or mixed glomerulo-tubular proteinuria. The exclusively renal excretion of albumin and transferrin characterizes the selective proteinuria which occurs, for example, in minimal-change nephropathy. In non-selective proteinuria, IgG can also be detected in the urine. This form of proteinuria can be found, for example, in renal amyloidosis, but also in an advanced state of diabetic nephropathy. Tubular proteinuria is based on tubolo-interstitial diseases which affect reabsorptive processes, which results in the excretion of low-molecular weight proteins. Clinically, tubular proteinuria is of importance, in particular, when associated with other defects of the proximal tubule. Tubular proteinurias occur, inter alia, in diseases such as hereditary tubulopathy, renal-tubular azidosis, interstitial nephritis induced by bacteria or medicaments, acute renal failure, heavy metal poisoning, Bence-Jones nephropathy and in the postsurgical phase of kidney transplantation. Mixed glomerulo-tubular proteinuria is often based on primary glomerular diseases with pronounced secondary interstitial changes.

Therefore, in a preferred embodiment of the invention, the renal diseases involving proteinuria are, in particular, minimal-change nephropathy, other glomerulopathies, kidney amyloidosis, hereditary tubulopathy, renal-tubular azidosis, interstitial nephritis induced by bacteria or medicaments, acute renal failure, Bence-Jones nephropathy and the postsurgical phase of kidney transplantation.

In the context of the present invention, “diabetes mellitus” means various forms of glycose metabolic disorders with different etiologies and symptoms. A common characteristic is a relative or absolute insulin deficiency. Diabetes mellitus diseases are characterized by a permanent increase of blood glucose level (hyperglycemia) or by a mistimed utilization of supplied glucose. Diabetes mellitus is subdivided into type I (Insulin-dependent; IDDM) and type II (non-insulin-dependent; NIDDM).

Diabetes-specific and diabetes-associated chronic complications include microangiopathy, such as retinopathy, nephropathy and neuropathy, polyneuropathy, diabetic foot, disorders of the skeletal, supporting and connective tissue as well as macroangiopathy, especially coronary heart disease, cerebral circulation disorder and peripheral arterial occlusive disease.

“Diabetic retinopathy” means a microangiopathy of the eye-ground occurring in diabetes mellitus. Forms of diabetic retinopathy are non-proliferative retinopathy (background retinopathy), such as retinal hemorrhages, microaneurysms, hard exudates, retinal edema with loss of visual acuity, as well as proliferative retinopathy, in which there is additional occurrence of cotton-wool spots and angioneogenesis on and in front of the retina with vitreous hemorrhage due to retinal ischemia from vascular occlusion. Proliferative retinopathy may result in traction retinal detachment, neovascular glaucoma and blindness.

In the context of the present invention, “diabetic nephropathy”, which is also referred to as diabetic glomerulosclerosis, means damage to the glomerular kidney capillaries. Clinically, diabetic nephropathy is manifested by proteinuria, hypertension, edemas, a diffuse widening of the basal membrane, mesangial hypertrophy and later nodular swellings in the loops of the glomerulus with constriction of the vascular lumen as well as fibrinoid depositions in the capillary wall and microaneurysms.

In the context of the present invention, “diabetic neuropathy” means a disease of the peripheral nerves. In particular, it means symmetric distal sensomotoric polyneuropathy and autonomous neuropathy. Peripheral neuropathy is typically manifested in the lower extremities, beginning with the feet, and proceeds towards proximal and not infrequently also affects the arms. The symptoms vary significantly, and complaints such as pain, numbness and paresthesia often result in exacerbation.

According to the invention, “cardiovascular complications in diabetes” means cardiovascular and vascular diseases, especially peripheral occlusive disease, coronary heart disease, myocardial infarction and stroke, which occur as a consequence of diabetes mellitus.

In the context of the present invention, “hypertension” means high blood pressure or hypertensive heart disease which is characterized by a permanent increase of blood pressure to values of more than 140 mm Hg systolic and more than 90 mm Hg diastolic. According to the Invention, “cardiovascular complications associated with hypertension” means cardiovascular and vascular diseases, especially peripheral occlusive disease, coronary heart disease, myocardial infarction and stroke, which occur as a consequence of hypertension.

In the context of the present invention, “hypercholesterolemia” means an increased cholesterol level in the blood, wherein the hypercholesterolemia may occur primarily or secondarily as a consequence of diabetes. Hypercholesterolemia is a risk factor of atherosclerosis. According to the invention, “cardiovascular complications associated with hypercholesterolemia” means cardiovascular and vascular diseases, especially peripheral occlusive disease, coronary heart disease, myocardial infarction and stroke, which occur as a consequence of hypercholesterolemia.

In the context of the present invention, a “protein kinase C” or “PKC” means a family of proteins which plays an essential role in signal transmission, the PKC proteins serving intracellular regulatory functions by the phosphorylation of substrates, such as enzymes, transcription factors and/or cytoskeleton proteins. For example, activation of the PKC proteins results in an activation of further protein kinases including mitogen-activated protein kinase (MAPK), which are thus substrates of the PKC proteins. Protein kinase C proteins are the main phorbol ester receptors. The protein kinase C family of proteins comprises at least twelve isoforms in mammal cells which are subdivided into three different subfamilies. The so-called conventional protein kinase C isoforms (cPKC) comprise the isoforms PKC-α, PKC-βI and its splice variant βII as well as PKC-gamma. The so-called novel protein kinase isoforms (nPKC) comprise the isoforms PKC-delta, PKC-epsilon, PKC-eta and PKC-theta. The so-called atypical protein kinase C isoforms (aPKC) comprise the Isoforms PKC-zeta and PKC-lambda (also known as PKC-iota). Further isoforms are PKC-mu (also referred to as protein kinase D) and the PKC-related kinases (PRK) which may be separate subfamilies (Toker, Frontiers in Biosciences, 3 (1998), d1134-1147). The PKC isoforms are distinguished in both their amino acid sequences and the nucleic acid sequences coding for the amino acid sequences (Coussens et al., Sciences, 233 (1986), 859-866). The PKC proteins all have a domain structure. Their cellular expression patterns, their mechanisms of activation and their substrate specificities are also different.

The majority of protein kinase C isoforms are not membrane-bound before activation and is diffusely distributed in the cytoplasm. The activation of the activity of each isoform by treating the cells with the phorbol compound 12-O-tetradecanoylphorbol-13-acetate results in isozyme-specific changes of cell morphology as well as in a rapid and selective redistribution of the different PKC isozymes into different subcellular structures. The protein kinase C-α isoform becomes enriched, in particular, in the endoplasmic reticulum and at the cellular edge, while the PKC βII isoform is enriched in the actin-rich microfilaments of the cytoskeleton. The substrate specificity of the PKC isoforms is mediated at least partially by the subcellular distribution of the activated protein kinase C isozymes.

“Protein kinase C-α” means a protein which is activated by calcium ions and diacylglycerol, the activated protein kinase C-α becoming enriched, in particular, in the endoplasmic reticulum and at the cellular edge. The amino acid sequence of PKC-α and the nucleic acid sequence coding for PKC-α are described in Coussens et al., Sciences, 233 (1986), 859-866. The PKC-α protein has a similar domain structure as the remaining cPKC proteins. The protein comprises a pseudosubstrate domain, a cysteine-rich region, a calcium-binding domain and a catalytic domain. PKC-α can be activated by diacylglycerol, phorbol ester, phosphatidylserine and calcium.

In the context of the present invention, “agents which reduce or inhibit the expression of protein kinase C-α” means those agents which completely prevent or at least reduce the synthesis of a functional PKC-α protein under both in-vitro and in-vivo conditions, said reduction or inhibition concerning the transcription of the DNA sequence coding for PKC-α into a complementary mRNA sequence, the processing of the mRNA, the translation of the mRNA into a polypeptide chain, the processing of the polypeptide and/or posttranslational modifications of the polypeptide. Thus, the use of agents which reduce or inhibit the expression of protein kinase C-α may cause that either no functional, for example, activatable, PKC-α protein is prepared at all, or that the amount of the produced functional, for example, activatable, PKC-α protein is reduced. However, the use of agents which reduce or inhibit the expression of protein kinase C-α may cause that a non-functional, for example, non-activatable, PKC-α protein or an only partially functional PKC-α protein is produced.

In the context of the present invention, “agents which reduce or inhibit the activity of protein kinase C-α” means those agents which can completely or partially eliminate the biological activity of the functional PKC-α protein under both In-vitro and in-vivo conditions. The complete or partial inactivation of the PKC-α protein may be effected, for example, by a direct interaction of the agent employed with the PKC-α protein. The direct interaction between the agent and PKC-α protein can be effected, for example, by covalent or non-covalent binding. The interaction between the agent and PKC-α protein may also cause, for example, chemical changes in the protein kinase, which results in a loss of the biological activity of the protein kinase. The interaction may also lead, for example, to a specific degradation of PKC-α. However, agents which reduce or Inhibit the activity of protein kinase C-α may also be those which modify or eliminate or bind to specific substrates, target structures or target molecules of PKC-α in such a way that the biological activity of PKC-α is reduced or completely suppressed. Agents which reduce or inhibit the activity of protein kinase C-α may also be those which prevent the translocation of PKC-α into the endoplasmic reticulum or to the edge of the cell after activation, for example, activation by phorbol treatment, so that PKC-α cannot interact with its specific substrates, target structures or target molecules.

In a particularly preferred embodiment of the invention, the agents employed according to the invention are agents which specifically reduce or inhibit the expression and/or activity of PKC-α, but not the expression and/or activity of other PKC isoforms, for example, PKC-β.

According to the invention, the agents which specifically reduce or inhibit the expression and/or activity of PKC-α are selected from the group consisting of nucleic acids which reduce or inhibit the expression of the protein kinase C-α gene, vectors containing said nucleic acid, host cells containing said vectors, substances which inhibit or reduce the expression of protein kinase C-α, substances which inhibit the translocation of protein kinase C-α, antagonists of protein kinase C-α activity, and inhibitors of protein kinase C-α activity.

According to the invention, the nucleic acid employed is preferably selected from the group consisting of

a) a nucleic acid coding for human protein kinase C-α, or a fragment thereof;
b) a nucleic acid which is complementary to the nucleic acid according to a), or a fragment thereof;
c) a nucleic acid which is obtainable by substitution, addition, inversion and/or deletion of one or more bases of a nucleic acid according to a) or b), or a fragment thereof; and
d) a nucleic acid which has more than 80% homology with a nucleic acid according to a) through c), or a fragment thereof.

In the context of the present invention, a “nucleic acid coding for protein kinase C-α, or a fragment thereof” means a nucleic acid which codes for a PKC-α protein or a fragment thereof which comprises the functional domains, especially the pseudosubstrate domain, the cysteine-rich region, the calcium-binding domain and a catalytic domain of native protein kinase C-α. In a preferred embodiment of the invention, the nucleic acid used according to the invention codes for human PKC-alpha or parts thereof.

In the context of the invention, “homology” means a sequence Identity of at least 80%, preferably at least 85% and more preferably more than 90%, 95%, 97% and 99%. Thus, the term “homology” which is known to the skilled person designates the degree of relationship between two or more nucleic acid molecules, which is determined by the agreement between the sequences.

The nucleic acid used according to the invention may be a DNA or RNA sequence, especially in a linear form. The nucleic acid may be isolated from natural sources, for example, from eukaryotic tissues, preferably mammal tissues, more preferably from human tissues, or prepared synthetically.

According to the invention, it is provided, in particular, that the nucleic acid used as the agent, if inserted in a vector, especially an expression vector, can inhibit the expression of the gene of human protein kinase C-α in a host cell in antisense orientation to a promoter. When the nucleic acid employed according to the invention is inserted in a vector in antisense orientation, i.e., when an antisense construct of the nucleic acid employed according to the invention is employed, the nucleic acid will be transcribed as an antisense nucleic acid. Then, when the native PKC-alpha gene of the cell is transcribed, the antisense transcript produced of the nucleic acid used according to the invention can bind through Watson-Crick base pairing to the mRNA transcript of the native protein kinase C-α gene which is in sense orientation to form a duplex structure. In this way, the translation of the mRNA of the native PKC-α gene into a polypeptide is selectively suppressed, and the expression of the native PKC-alpha is specifically inhibited without inhibiting the expression of other cellular PKC isoforms.

In a preferred embodiment of the invention, it is provided that the nucleic acid used for the production of antisense constructs does not comprise the entire sequence coding for PKC-alpha, but only fragments thereof. Such fragments comprise at least 10 nucleotides, preferably at least 50 nucleotides, more preferably at least 200 nucleotides, wherein the nucleotide regions of the sequence coding for PKC-alpha which are spanned by the fragments are selected in such a way that, when the fragments are expressed in antisense orientation in a cell, specific inhibition of the expression of PKC-alpha, especially human PKC-alpha, occurs, but not inhibition of other PKC isoforms, for example, the PKC-beta isoforms.

According to the invention, it is provided that the above mentioned nucleic acid or the suitable fragment thereof. Is inserted in a vector under the control of at least one expression regulating element, wherein the nucleic acid or its fragment is inserted in an antisense orientation with respect to said expression regulating elements. Thus, after the vector has been introduced into a cell, for example, a mammal cell, especially a human cell, the nucleic acid or its fragment can be expressed in antisense orientation and thus efficiently inhibit the expression of the native PKC-alpha of the cell. Preferably, the vector is a plasmid, cosmid, bacteriophage or virus.

Therefore, the present invention also relates to a vector which comprises a nucleic acid sequence coding for PKC-alpha or a fragment thereof under the functional control of at least one expression regulating element, wherein the nucleic acid or its fragment is inserted in an antisense orientation with respect to said expression regulating element. Said expression regulating element is, in particular, a promoter, a ribosome binding site, a signal sequence or a 3′ transcription terminator.

Another embodiment of the invention relates to a host cell which contains an above described vector. In particular, the host cell is a mammal cell, preferably a human cell. In a particularly preferred form, the human cell is an adult stem cell.

In a preferred embodiment of the invention, synthetically prepared antisense oligonucleotides which comprise at least 10 nucleotides, preferably at least 50 nucleotides, more preferably at least 200 nucleotides, are employed for inhibiting the expression of PKC-alpha. Such antisense oligonucleotides can be directly employed for inhibiting PKC-alpha expression, i.e., need not be inserted into a vector and expressed under cellular conditions. In a particularly preferred embodiment, these PKC-α-specific antisense oligonucleotides are the product ISIS 3521 from Isis Pharmaceuticals, which is a strong selective inhibitor of protein kinase alpha expression. In a further particularly preferred embodiment of the invention, the PKC-α-specific antisense oligonucleotides employed according to the invention are the antisense oligodeoxynucleotides described by Busutti et al., J. Surg. Pathol., 63 (1996), 137-142.

According to the invention, the above mentioned nucleic acids, the vectors containing such nucleic acids or the host cells containing such vectors can be equally employed as agents for the treatment and/or prevention of vascular diseases, cardiovascular diseases, renal diseases involving proteinuria, late effects and/or cardiovascular complications associated with diabetes mellitus, cardiovascular complications associated with hypertension, and/or cardiovascular complications associated with hypercholesterolemia, for example, within the scope of gene therapy.

In another preferred embodiment of the invention, it is provided that an activator of protein kinase C-α is employed for inhibiting or reducing the expression of protein kinase alpha. Preferably, said activator is a phorbol compound, especially 12-o-tetradecanoylphorbol-13-acetate (TPA) or phorbol-12,13-dibutyrate (PDBu). It is known that the incubation of cells with, for example, PDBu over a period of 16 h to 24 h results in a complete down regulation of PKC-alpha (Busutti et al., J. Surg. Res., 63 (1996), 137-142). Also, it is known that treatment with a higher TPA concentration, for example, 1.6 μM, completely inhibits PKC-alpha expression. Therefore, according to the invention, the treatment of afflicted tissues with phorbol esters in a concentration of preferably more than 1.6 μM over a period of at least 15 h is provided in order to block the expression of PKC-alpha in the respective tissues or organs partially or completely.

In another preferred embodiment, the use of an inhibitor for inhibiting or reducing the activity of protein kinase α is provided. In the context of the present invention, “inhibitor” means a substance which competitively inhibits the biological activity of protein kinase C-α, allosterically changes the spatial structure of PKC-α, or inhibits PKC-α by substrate inhibition.

In a preferred embodiment of the invention, the inhibitor is an antibody which specifically reacts with protein kinase C-α. “Antibody” means polypeptides which are essentially coded for by an immunoglobulin gene or genes or fragments thereof and which are able to specifically bind and recognize an analyte, i.e., an antigen. The binding of the antibody to PKC-α inhibits the biological activity of the latter. According to the invention, the antibodies against the protein kinase C-α may be employed as intact immunoglobulins or as a number of fragments produced by cleavage with various peptidases. The term “antibodies” as used according to the invention also relates to modified antibodies, for example, oligomeric antibodies, reduced antibodies, oxidized antibodies and labeled antibodies. The term “antibody” also comprises antibody fragments prepared either by modifying the whole antibody, or de novo with the use of recombinant DNA methods. Therefore, the term “antibody” comprises both intact molecules and fragments thereof, such as Fab, F(ab′)₂ and FV, which can bind to the epitopic determinants. These antibody fragments retain the ability to bind selectively to the corresponding antigen. Methods for the preparation of antibodies or fragments thereof are known in the prior art.

In a preferred embodiment of the invention, the antibody employed according to the invention for inhibiting the activity of protein kinase C-α is a monoclonal or polyclonal antibody. According to the invention, the antibody may also be a humanized antibody. In a particularly preferred embodiment of the Invention, the antibodies used for inhibiting the activity of protein kinase C-α are those as described by Goodnight et al., J. Biol. Chem., 270 (1995), 9991-10001.

In a further preferred embodiment of the invention, it is provided that the inhibitor employed according to the application for inhibiting PKC-alpha changes the phosphorylation state of protein kinase C-α and thus inhibits or at least reduces the activity of PKC-alpha. From Tasinato et al., Biochem. J., 334 (1998), 243-249, it is known that alpha-tocopherol can inactivate the cellular protein kinase C-alpha by changing the phosphorylation state of PKC-alpha. Therefore, in a particularly preferred embodiment of the invention, the use of alpha-tocopherol for inhibiting the activity of PKC-alpha and thus for the treatment and/or prevention of vascular diseases, cardiovascular diseases, renal diseases involving proteinuria, diabetic late effects and/or cardiovascular complications in patients with diabetes mellitus, cardiovascular complications in patients with hypertension and/or cardiovascular complications in patients with hypercholesterolemia is provided.

In a further preferred embodiment of the invention, the use of antagonists of PKC-alpha for the treatment and/or prevention of vascular diseases, cardiovascular diseases, renal diseases involving proteinuria, diabetic late effects and/or cardiovascular complications in patients with diabetes mellitus, cardiovascular complications in patients with hypertension and/or cardiovascular complications in patients with hypercholesterolemia is provided. In the context of the present invention, “antagonist” means a substance which competes with PKC-alpha for the binding to a PKC-alpha-specific substrate, but without causing the same effect as PKC-alpha after binding to the substrate. The term “antagonists” also includes substances which are adapted to an inactive conformation of a PKC-alpha-specific substrate due to their structure and therefore prevent the activation of the substrate by PKC-alpha.

In a preferred embodiment of the invention, a derivative of PKC-alpha which can bind to the substrates of the native PKC-alpha, but without causing the same biological effect as native PKC-alpha after binding thereto, is employed as the antagonist for inhibiting the PKC-alpha activity.

In the context of the present invention, “derivatives” means functional equivalents or derivatives of protein kinase C-α which are obtained by substituting atoms or molecular groups or residues while retaining the PKC-α basic structure, and/or whose amino acid sequences differ from the naturally occurring sequence of human or animal PKC-α molecules in at least one position, but which essentially have a high degree of homology on the amino acid level. According to the invention, the term “derivative” also includes fusion proteins in which functional domains of another protein, for example, another PKC-α inhibitor, are present in the N-terminal or C-terminal portions.

The differences between a derivative and native PKC-α may arise, for example, from mutations, such as deletions, substitutions, insertions, additions, base exchanges and/or recombinations of the nucleotide sequences coding for the PKC-α amino acid sequences. Of course, these may also be naturally occurring sequence variations, for example, sequences from another organism or sequences mutated in a natural way, or mutations which have been purposefully introduced into the corresponding sequences by usual means known in the art, for example, chemical agents and/or physical agents.

In another preferred embodiment of the invention, an analogue of PKC-alpha is employed as the antagonist for inhibiting the PKC-alpha activity. In the context of the present invention, “analogues” of protein kinase C means compounds which do not have an amino acid sequence identical with that of protein kinase C-α, but whose three-dimensional structure is highly similar to that of protein kinase C-α. The analogues of PKC-α employed according to the invention preferably have substrate specificity properties similar to those of PKC-α, i.e., they can bind to the PKC-alpha-specific substrates, but preferably lack the catalytic properties of PKC-α. Therefore, the protein kinase C-α analogues employed according to the invention may be, for example, compounds which contain the amino acid residues responsible for the binding of protein kinase C-α to PKC-α substrates in a suitable conformation and are therefore able to mimic the essential properties of the binding region of protein kinase C-α, but without possessing the same catalytic properties as protein kinase C-α.

In another embodiment of the invention, it is provided that, for the treatment and/or prevention of vascular diseases, cardiovascular diseases, renal diseases involving proteinuria, late effects and/or cardiovascular complications in patients with diabetes mellitus, cardiovascular complications in patients with hypertension, and/or cardiovascular complications in patients with hypercholesterolemia, agents are employed which reduce or inhibit not only the expression and/or activity of protein kinase C-α (PKC-α), but at the same time the expression and/or activity of protein kinase C-β (PKC-β). In J. Invest. Dermatol., 117 (2001), 605-611, Takahashi and Kamimura describe that the immunosuppressant cyclosporine A reduces the expression of the protein kinase C isoforms alpha, beta I and beta II at the same time. Therefore, in a particularly preferred embodiment of the invention, the use of cyclosporine A for reducing the expression of PKC-alpha and PKC-beta and thus for the treatment of the above mentioned diseases is provided.

In another preferred embodiment of the invention, it is provided that the agent which specifically reduces or inhibits the expression and/or activity of protein kinase C-α is used in combination with an agent which specifically reduces or inhibits the expression and/or activity of protein kinase C-β. According to the invention, “protein kinase C-β” includes both protein kinase C-βI and the splice variant βII.

According to the invention, it is provided that the agent which reduces or inhibits the expression and/or activity of protein kinase C-β Is selected from the group consisting of nucleic acids which reduce or inhibit the expression of the protein kinase C-β gene, vectors containing said nucleic acid, host cells containing said vectors, substances which inhibit or reduce the expression of protein kinase C-β, substances which inhibit the translocation of protein kinase C-β, antagonists of protein kinase C-β activity, and inhibitors of protein kinase C-β activity.

In a preferred embodiment of the Invention, the nucleic acid to be employed as an agent is selected from the group consisting of

a) a nucleic acid coding for human protein kinase C-β, or a fragment thereof;
b) a nucleic acid which is complementary to the nucleic acid according to a), or a fragment thereof;
c) a nucleic acid which is obtainable by substitution, addition, inversion and/or deletion of one or more bases of a nucleic acid according to a) or b), or a fragment thereof; and
d) a nucleic acid which has more than 80% homology with a nucleic acid according to a) through c), or a fragment thereof.

In a preferred embodiment of the invention, the nucleic acid used according to the invention codes for human PKC-alpha or parts thereof.

The nucleic acid used according to the invention may be a DNA or RNA sequence, especially in a linear form. The nucleic acid may be isolated from natural sources, for example, from eukaryotic tissues, preferably mammal tissues, more preferably from human tissues, or prepared synthetically.

According to the invention, it is provided, in particular, that the nucleic acid used as the agent, if inserted in a vector, especially an expression vector, can inhibit the expression of the gene of human protein kinase C-β in a host cell in antisense orientation to a promoter. When the nucleic acid employed according to the invention is inserted in a vector in antisense orientation, i.e., when an antisense construct of the nucleic acid employed according to the invention is employed, the nucleic acid will be transcribed as an antisense nucleic acid. Then, when the native PKC-β gene of the cell is transcribed, the antisense transcript produced of the nucleic acid used according to the invention can bind to the mRNA transcript of the native protein kinase C-β gene which is in sense orientation to form a duplex structure. In this way, the translation of the mRNA of the native PKC-β gene into a polypeptide is selectively suppressed, and the expression of the native PKC-β is specifically inhibited without inhibiting the expression of other cellular PKC isoforms.

In a preferred embodiment of the invention, it is provided that the nucleic acid used for the production of antisense constructs does not comprise the entire sequence coding for PKC-β, but only fragments thereof. Such fragments comprise at least 10 nucleotides, preferably at least 50 nucleotides, more preferably at least 200 nucleotides, wherein the nucleotide regions of the sequence coding for PKC-β which are spanned by the fragments are selected in such a way that, when the fragments are expressed in antisense orientation in a cell, specific inhibition of the expression of PKC-β, especially human PKC-β, occurs, but not inhibition of other PKC isoforms.

According to the invention, it is provided that the above mentioned nucleic acid or the suitable fragment thereof is inserted in a vector under the control of at least one expression regulating element, wherein the nucleic acid or its fragment is inserted in an antisense orientation with respect to said expression regulating elements. Thus, after the vector has been introduced into a cell, for example, a mammal cell, especially a human cell, the nucleic acid or its fragment can be expressed in antisense orientation and thus efficiently inhibit the expression of the native PKC-p of the cell. Preferably, the vector is a plasmid, cosmid, bacteriophage or virus.

Therefore, the present invention also relates to a vector which comprises a nucleic acid sequence coding for PKC-β or a fragment thereof under the functional control of at least one expression regulating element, wherein the nucleic acid or its fragment is inserted in an antisense orientation with respect to said expression regulating element. Said expression regulating element is, in particular, a promoter, a ribosome binding site, a signal sequence or a 3′ transcription terminator.

Another embodiment of the invention relates to a host cell which contains an above described vector. In particular, the host cell is a mammal cell, preferably a human cell. In a particularly preferred form, the human cell is an adult stem cell.

In a preferred embodiment of the invention, synthetically prepared antisense oligonucleotides which comprise at least 10 nucleotides, preferably at least 50 nucleotides, more preferably at least 200 nucleotides, are employed for inhibiting the expression of PKC-β. Such antisense oligonucleotides can be directly employed for inhibiting PKC-β expression, I.e., need not be inserted into a vector and expressed under cellular conditions.

In another preferred embodiment of the invention, it is provided that an antibody which specifically reacts with protein kinase C-β or a suitable fragment thereof is employed for inhibiting the activity of protein kinase C-β. According to the invention, the antibody may be a monoclonal or polyclonal antibody. The antibody employed according to the invention may also be a humanized antibody.

In a further preferred embodiment of the invention, it is provided that a substance which changes the phosphorylation state of protein kinase C-β is employed for inhibiting the activity of protein kinase C-β.

In another preferred embodiment of the invention, it is provided that a derivative or analogue of protein kinase C-β which acts as an antagonist of PKC-β is employed for inhibiting the activity of protein kinase C-β. Preferably, the derivatives or analogues of PKC-β employed according to the invention are substances which compete with the native PKC-β for the binding to PKC-p-specific substrates, but without causing the same effect as PKC-β after binding to the substrates.

In a particularly preferred embodiment of the invention, the compounds as described in the documents U.S. Pat. No. 5,491,242, U.S. Pat. No. 5,661,173, U.S. Pat. No. 5,481,003, U.S. Pat. No. 5,668,152, U.S. Pat. No. 5,672,618, WO 95/17182, WO 95/35294 and WO 02/are employed for specifically inhibiting and reducing the expression and/or activity of protein kinase C-β.

Another preferred embodiment of the invention relates to the use of agents which specifically reduce or inhibit the expression and/or activity of protein kinase C-α (PKC-α) for the preparation of a pharmaceutical composition for the treatment and/or prevention of coronary heart disease, myocardial infarction, peripheral occlusive disease, stroke, renal diseases involving proteinuria, diabetic late effects and/or cardiovascular complications in patients with diabetes mellitus, cardiovascular complications in patients with hypertension, and cardiovascular complications in patients with hypercholesterolemia. According to the invention, the cardiovascular complications are preferably coronary heart disease, myocardial infarction, peripheral occlusive disease and stroke. The diabetic late effects are, in particular, diabetic retinopathy, diabetic neuropathy and diabetic nephropathy.

In the context of the present invention, a “pharmaceutical composition” or a “medicament” means a mixture used for diagnostic, therapeutic and/or prophylactic purposes, i.e., a mixture which promotes or restores the health of a human or animal body, which comprises at least one natural or synthetically prepared active ingredient which causes the therapeutic effect. The pharmaceutical composition may be both a solid and a liquid mixture. For example, a pharmaceutical composition comprising the active ingredient may contain one or more pharmaceutically acceptable components. In addition, the pharmaceutical composition may comprise additives usually employed in the art, for example, stabilizers, production agents, separating agents, disintegrants, emulsifiers or other materials usually employed for the preparation of pharmaceutical compositions.

According to the invention, in particular, the use of agents which specifically reduce or inhibit the expression and/or activity of protein kinase C-α (PKC-α) as an active ingredient for the preparation of a medicament for the therapy and/or prophylaxis of the above mentioned diseases is provided. In a preferred embodiment of the invention, the agents employed for the preparation of pharmaceutical compositions are selected from the group consisting of nucleic acids which reduce or inhibit the expression of the protein kinase C-α gene, vectors containing said nucleic acid, host cells containing said vectors, substances which inhibit or reduce the expression of protein kinase C-α, substances which inhibit the translocation of protein kinase C-α, antagonists of protein kinase C-α activity, and inhibitors of protein kinase C-α activity.

More preferably, the agents employed for the preparation of the pharmaceutical composition according to the invention are antisense oligonucleotides of the gene coding for protein kinase C-α, tocopherol, phorbol compounds, derivatives of protein kinase C-α, or analogues of protein kinase C-α.

In a preferred embodiment of the invention, it is provided that the pharmaceutical composition is used for parenteral, especially intravenous, intramuscular, intracutaneous or subcutaneous administration. Preferably, the medicament containing the agents employed according to the invention is in the form of an injection or infusion.

In another embodiment of the invention, it is provided that the pharmaceutical composition containing the agents employed according to the invention is administered orally. For example, the medicament is administered in a liquid dosage form, such as a solution, suspension or emulsion, or in a solid dosage form, such as a tablet.

Therefore, the present invention also relates to pharmaceutical compositions for the prevention and/or treatment of coronary heart disease, myocardial infarction, peripheral occlusive disease, stroke, renal diseases involving proteinuria, diabetic late effects and/or cardiovascular complications in patients with diabetes mellitus, cardiovascular complications in patients with hypertension, and cardiovascular complications in patients with hypercholesterolemia, comprising at least one agent which specifically reduces or inhibits the expression and/or activity of protein kinase C-α (PKC-α) as an active ingredient.

In a preferred embodiment, the agents contained in the pharmaceutical composition are selected from the group consisting of nucleic acids which reduce or inhibit the expression of the protein kinase C-α gene, vectors containing said nucleic acid, host cells containing said vectors, substances which inhibit or reduce the expression of protein kinase C-α, substances which inhibit the translocation of protein kinase C-α, antagonists of protein kinase C-α activity, and inhibitors of protein kinase C-α activity.

More preferably, the pharmaceutical composition according to the invention contains antisense oligonucleotides of the gene coding for protein kinase C-α, tocopherol, phorbol compounds, derivatives of protein kinase C-α, or analogues of protein kinase C-α.

In another preferred embodiment of the invention, the pharmaceutical composition according to the invention contains at least one further active ingredient. In particular, said further active ingredient is an agent which specifically reduces or inhibits the expression and/or activity of protein kinase C-β.

Preferably, the agent which specifically reduces or inhibits the expression and/or activity of protein kinase C-β is selected from the group consisting of nucleic acids which reduce or inhibit the expression of the protein kinase C-β gene, vectors containing said nucleic acid, host cells containing said vectors, substances which inhibit or reduce the expression of protein kinase C-β, substances which inhibit the translocation of protein kinase C-β, antagonists of protein kinase C-β activity, and inhibitors of protein kinase C-β activity.

Further advantageous embodiments of the invention can be seen from the dependent claims.

The invention is further Illustrated by means of the following Figures and Examples.

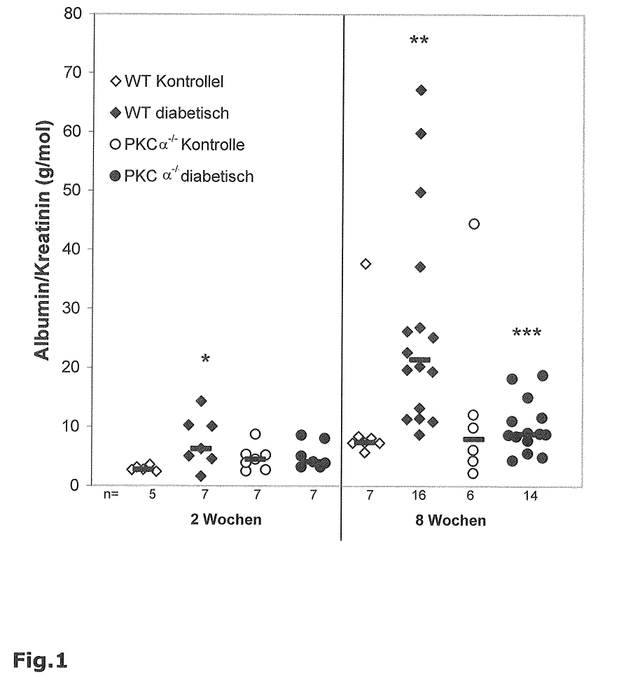

FIG. 1 shows the albumin excretion in the urine of PKC-alpha “knock out” mice with diabetes mellitus and without diabetes mellitus (control) and SV129 mice with diabetes and without diabetes (control). The albumin concentration was determined by using an indirect ELISA assay. The albumin values established were based on the creatinin concentration. The non-diabetic SV129 and PKC-α^{-/-} mice have a comparable albumin/creatinin quotient which is usually below 10 g/mol. In contrast, the quotient for diabetic SV129 mice is significantly higher (p=0.004). The values for the diabetic PKC-α^{-/-} mice are significantly lower than the values for the diabetic SV129 mice (p<0.001). The transverse bar indicates the median value. The significance was calculated by means of the Mann-Whitney U test.

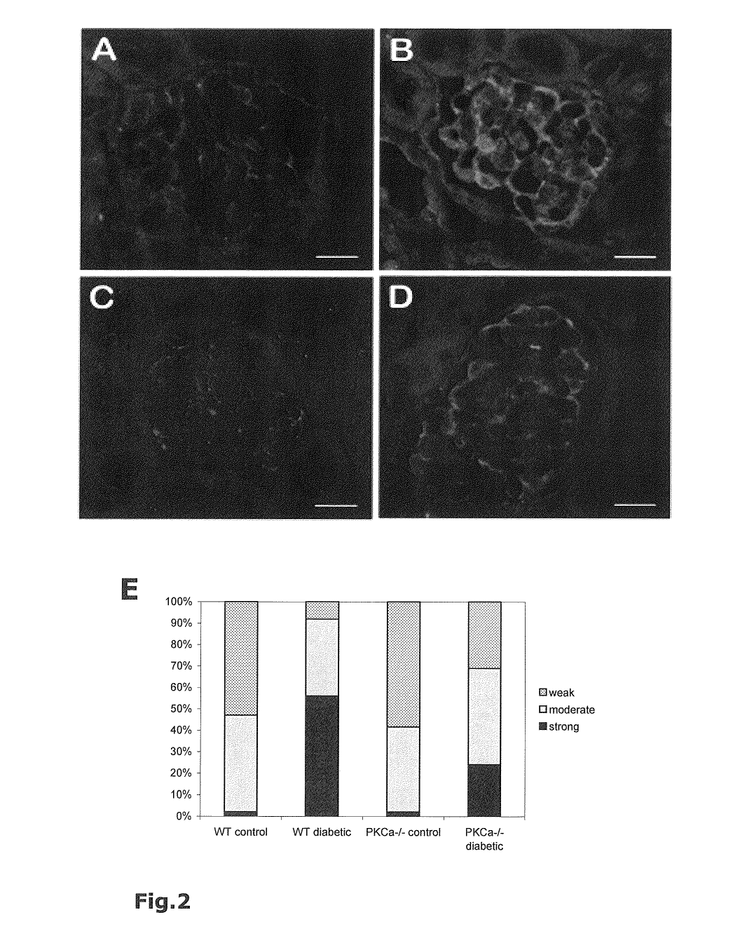

FIG. 2 shows the glomerular VEGF expression in PKC-alpha “knock out” mice with diabetes mellitus and without diabetes mellitus (control) and SV129 mice with diabetes and without diabetes (control). For each animal group, 40 glomeruli were evaluated semiquantitatively by means of immunohistochemical methods, and the values were divided into weak, medium and strong immunofluorescence. The significance was calculated by means of the Mann-Whitney U test. In diabetic animals, the VEGF expression is significantly higher as compared to control animals (p<0.001). However, the VEGF expression in diabetic SV129 animals is significantly higher as compared to diabetic PKC-alpha^{-/-} animals (p<0.001).

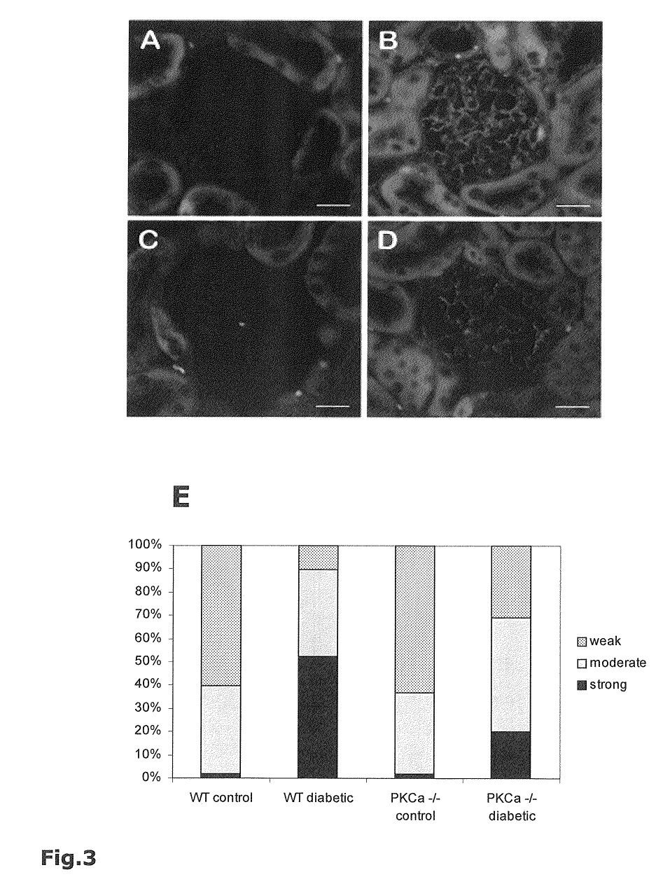

FIG. 3 shows the glomerular VEGF receptor II expression in PKC-alpha “knock out” mice with diabetes mellitus and without diabetes mellitus (control) and SV129 mice with diabetes and without diabetes (control). For each animal group, 40 glomeruli were evaluated semiquantitatively by means of immunohistochemical methods, and the values were divided into weak, medium and strong immunofluorescence. The significance was calculated by means of the Mann-Whitney U test. In diabetic animals, the VEGFR-II expression is significantly higher as compared to control animals (p<0.001). However, the VEGFR II expression in diabetic SV129 animals is significantly higher as compared to diabetic PKC-alpha^{-/-} animals (p<0.001).

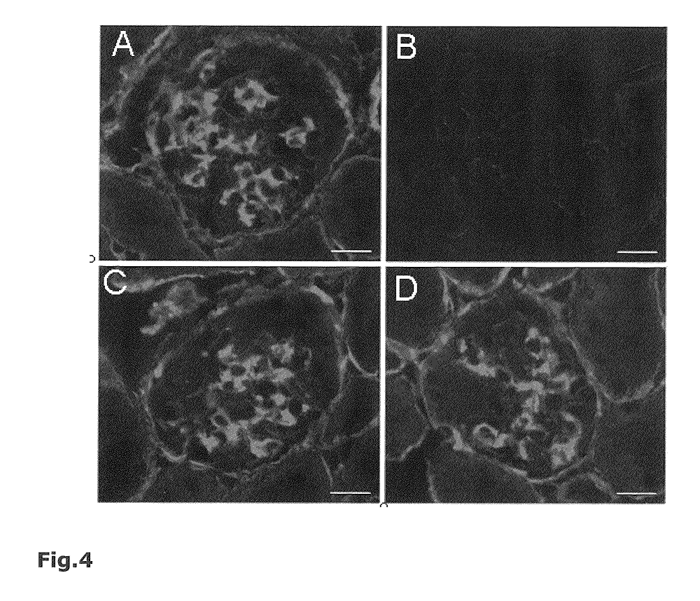

FIG. 4 shows the glomerular perlecan expression in PKC-alpha “knock out” mice with diabetes mellitus and without diabetes mellitus (control) and SV129 mice with diabetes and without diabetes (control). For each animal group, 40 glomeruli were evaluated semiquantitatively by means of immunohistochemical methods, and the values were divided into weak, medium and strong immunofluorescence. The significance was calculated by means of the Mann-Whitney U test. In diabetic SV129 animals, the perlecan expression is significantly lower as compared to SV129 control animals (p<0.001).

### EXAMPLE 1

### Experimental Diabetes Induction

With mice which were kept under standardized conditions at 22° C. with free access to feed and water, the following experiments were performed after approval by the animal protection authorities of Lower Saxony.

Before the start of the experiment, the blood sugar level from serum was determined for all animals. The results are shown in Table 1. In 16 SV129 control mice and 14 SV129 protein kinase C-alpha knock-out (PKC-α^{-/-}) mice, diabetes was induced by the injection of streptozotozin. Streptozotozin results in destruction of the insulin-producing islet cells in the pancreas. The resulting insulin deficiency causes permanently increased blood sugar levels, i.e., hyperglycemia, and thus diabetes mellitus. To produce the hyperglycemia, the animals were administered 125 mg each of streptozotozin per kg of body weight intraperitoneally on days 1 and 4. For such purposes, the streptozotozin was dissolved in a 50 mM Na citrate solution with a pH value of 4.5. For control, seven SV129 and six PKC-α^{-/-} mice were administered only the solvent intraperitoneally on days 1 and 4. Subsequently, a drop of blood was taken from the tail of the mice every two days in order to check the blood sugar level. The blood sugar measurement was performed by means of the Bayer Glucometer Elite® measuring device. Glucometer Elite Sensor® test strips were used for the determination.

On days 7-10, the animals to which streptozotozin had been administered were diabetic with blood sugar levels of above 350 mg/di. The starting values before streptozotozin injection were on average at 200 mg/dl. Animals which had obtained only the solvent did not exhibit any increase of the blood sugar levels and did not develop diabetes mellitus. Ten days after the first Injection, the animals were observed for further 8 weeks. During this time, the blood sugar was checked every two weeks to ensure that the diabetic animals were still diabetic. During this period, the sugar levels varied on average around 500-550 mg/dl for the diabetic animals and around 200 mg/dl for the non-diabetic animals.

After 8 weeks, the animals were anesthetized with the narcotic avertin. Under anesthesia, 400 μl of blood was subsequently taken from the venous plexus of the eye, and the whole bladder urine was taken from the bladder by a puncture with a 27 G needle. Subsequently, the kidneys were perfused with a Ringer lactate solution through the ventral aorta, and the kidneys were removed. Immediately thereafter, the animals were killed while under anesthesia. Subsequently, the blood sugar levels were determined from the serum. The blood sugar levels can be found in Table 1. As can be seen, the diabetic animals have about 2.5 to 3 times higher glucose levels than they had at the beginning of the experiment and also as compared with the non-diabetic control animals.

TABLE 1
Serum glucose in diabetic and non-diabetic mice before
the beginning and at the end of the experiment

| | Serum glucose before | Serum glucose |
|---|---|---|
| | the beginning | at the end |
| | of the experiment | of the experiment |
| | (mg/dl) | (mg/dl) |
| SV129 control (n = 7) | 205 +/− 40 | 223 +/− 43 |
| SV129 diabetic (n = 16) | 192 +/− 36 | 505 +/− 80* |
| PKC-α^{-/-} control (n = 6) | 223 +/− 27 | 197 +/− 21 |
| PKC-^{-/-} diabetic (n = 14) | 225 +/− 31 | 589 +/− 98* |

*p ≦ 0.001 as compared to non-diabetic control animals

### EXAMPLE 2

### Determination of Albumin Concentration

The development of albuminuria in patients with diabetes is a known phenomenon. Therefore, the albumin excretion in the urine of PKC-alpha “knock out” mice with diabetes mellitus and without diabetes mellitus (control) and SV129 mice with diabetes and without diabetes (control) was determined. For this purpose, the albumin concentration was determined in the collected urine. To determine the albumin concentration, an Indirect ELISA assay (Albuwell M® of Exocell Inc., Philadelphia, USA) was used. This ELISA assay is specific for murine albumin. The determination was effected in accordance with the manufacturer's Instructions. To be able to account for variations in the urine excretion, the albumin values determined were based on the creatinin level in the urine. The results are shown in FIG. 1. It was found that the non-diabetic SV129 and PKC-α^{-/-} mice have a comparable albumin/creatinin quotient which is usually below 10 g/mol. In contrast, the quotient for diabetic SV129 mice is significantly higher (p=0.004). The median value is 21.5 g/mol vs. 7.48 g/mol for non-diabetic SV129 control animals. In comparison, there is no significant increase of albuminuria in diabetic PKC-α^{-/-} mice. The albumin/creatinin quotient is always below 20 g/mol, and the median is at 10.2 g/mol. The median for the non-diabetic PKC-α^{-/-} control mice is at 8.5 g/mol. The values for the diabetic PKC-α^{-/-} mice is significantly lower than the values for the diabetic SV129 mice (p<0.001). The results are shown in FIG. 1.

### EXAMPLE 3

### Determination of VEGF and VEGF Receptor II Expression

As set forth above in Example 1, all the animals were killed when the experiment was over. Immediately thereafter, the kidneys were removed and frozen at −70° C. A further analysis of the removed kidneys showed a significant increase of the expression of the “vascular endothelial growth factor” (VEGF) and VEGF receptor II (VEGFR-II) in the renal corpuscles (glomeruli) of the diabetic control animals. The detection of VEGF and VEGFR-II expression was effected by immunohistochemical methods. Thus, the kidneys frozen at −70° C. were cryo-sliced to a thickness of 6 nm and then dried in air. Subsequently, the cryo-slices were fixed with cold acetone, dried in air and washed with tris buffer (TBS: 0.05 M tris buffer, 0.15 M NaCl, pH 7.6). The cryo-slices were subsequently incubated for 60 minutes in a moist chamber with a primary polyclonal “rabbit” antibody against murine VEGF (Santa Cruz, A-20) or VEGFR-II (Santa Cruz, C-1158). After washing anew with TBS, the slices were incubated with a Cy3-labeled secondary “anti-rabbit” antibody (Jackson ImmunoResearch Laboratories, 711-165-152) for 30 minutes at room temperature and again washed with TBS. Subsequently, the preparations were evaluated and photographed through a Zeiss Axioplan-2 microscope (Zeiss, Jena, Germany). In all animals, 40 renal corpuscles each were evaluated, and the fluorescence intensity was divided into strong, medium and weak. In diabetic SV129 control animals, a significant increase (p<0.001) of VEGF and VEGFR-II expression was found as compared to non-diabetic control animals. In comparison, the increase of the expression was significantly less pronounced in diabetic SV129 and PKC-α^{-/-} mice (p<0.001). The results are shown in FIGS. 2 and 3.

### EXAMPLE 4

### Determination of Perlecan Expression

Since the established difference in VEGF expression alone could not account for the difference in albuminuria, the expression of the heparan sulfate proteoglycan perlecan in the kidneys of diabetic and non-diabetic animals was examined by means of immunohistochemical methods. Cryo-slices of the kidneys were prepared and embedded as described in Example 3. A monoclonal rat antibody directed against murine perlecan (RDI Systems, A7L6) was used as the primary antibody. A Cy3-labeled donkey anti-rat antibody (Jackson ImmunoResearch Laboratories, 712-165-153) was used as the secondary antibody. The immunohistochemical examination of the slices gave the completely surprising result that perlecan was no longer or hardly any longer detectable in diabetic control animals (cf. FIG. 4). Thus, perlecan could be detected neither in the glomerulus nor in the vascular wall of arterioles. In contrast, the expression of perlecan was unchanged or but slightly reduced in SV129 and PKC-α^{-/-} mice. Since the lack of heparan sulfate is considered one of the main mediators in the development of proteinuria, it is to be considered that this result can account for the absence of albuminuria.

1. A method of treatment and/or prevention of cardiovascular diseases in patients comprising administering an effective amount of an inhibitor of PKC alpha activity, wherein the inhibitor competitively inhibits the biological activity of PKC alpha or allosterically changes the spatial structure of PKC alpha.

2-55. (canceled)

56. The method of claim 1, wherein said cardiovascular diseases which affect the filing state and tonus of the circulatory system and the output performance of the heart are selected from the group consisting of coronary heart disease, myocardial infarction and stroke.

57. The method of claim 1, wherein said agent is selected from the group consisting of at least one nucleic acid which reduces or inhibits the expression of the protein kinase C-α gene, a vector containing said nucleic acid, a host cell containing said vector, a substance which reduces or inhibits the expression of protein kinase C-α, a substance which inhibits the translocation of protein kinase C-α, an antagonist of protein kinase C-α activity, and an inhibitor of protein kinase C-α activity.

58. The method of claim 57, wherein said nucleic acid can inhibit the expression of the gene of human protein kinase C-α in a host cell in anti-sense orientation to a promoter.

59. The method of claim 57, wherein said nucleic acid is selected from the group consisting of
a) a nucleic acid coding for human protein kinase C-α, or a fragment thereof;
b) a nucleic acid which is complementary to the nucleic acid of group a), or a fragment thereof;
c) a nucleic acid which is obtainable by substitution, addition, inversion and/or deletion of one or more bases of a nucleic acid of group a) or b), or a fragment thereof; and
d) a nucleic acid which has more than 80% homology with a nucleic acid any of group a) through c), or a fragment thereof.

60. The method of claim 57, wherein said fragment of the nucleic acid of any of group a) through d) comprises at least 10 nucleotides.

61. The method of claim 57, wherein said nucleic acid is a DNA or a RNA.

62. The method of claim 57, wherein said nucleic acid or fragment thereof is inserted in a vector under the control of at least one expression regulating element in antisense orientation thereto.

63. The method of claim 62, wherein said vector is selected from the group consisting of a plasmid, a cosmid, a bacteriophage or a virus.

64. The method of claim 62, wherein said expression regulating element is selected from the group consisting of a promoter, a ribosome binding site, a signal sequence or a 3′ transcription terminator.

65. The method of claim 62, wherein said vector is contained in a host cell.

66. The method of claim 65, wherein said host cell is a mammalian cell.

67. The method of claim 57, wherein said substance which inhibits or reduces the expression of protein kinase C-α is an activator of protein kinase C-α.

68. The method of claim 67, wherein said activator is a phorbol compound.

69. The method of claim 57, wherein said inhibitor of protein kinase C-α activity changes the phosphorylation state of protein kinase C-α.

70. The method of claim 69, wherein said inhibitor is tocopherol.

71. The method of claim 1, wherein the inhibitor of protein kinase C-α is an agent which reduces or inhibits the expression and/or activity of protein kinase C-β.

72. The method of claim 1, wherein the inhibitor of protein kinase C-α is administered in combination with an agent which reduces or inhibits the expression and/or activity of protein kinase C-β.

73. The method of claim 72, wherein said agent which reduces or inhibits the expression and/or activity of protein kinase C-β is selected from the group consisting of at least one nucleic acid which reduces or inhibits the expression of the protein kinase C-β gene a vector containing said nucleic acid, a host cell containing said vector, a substance which reduces or inhibits the expression of protein kinase C-β, a substance which inhibits the translocation of protein kinase C-β, an antagonist of protein kinase C-β activity, and an inhibitor of protein kinase C-β activity.

74. The method of claim 73, wherein said inhibitor of protein kinase C-β activity changes the phosphorylation state of protein kinase C-β.
